(19) United States
(12) Patent Application Publication
(10) Pub. No.: US 2023/0194868 A1
(43) Pub. Date: Jun. 22, 2023

(54) WELDING MASK PRESENTATION DEVICE

(71) Applicant: Daryl Svendson, Bella Coola (CA)

(72) Inventor: Daryl Svendson, Bella Coola (CA)

(21) Appl. No.: 17/555,305

(22) Filed: Dec. 17, 2021

(51) Int. Cl. G02B 27/01; A61F 9/06

(52) U.S. Cl. CPC G02B 27/0172; A61F 9/06; G02B 2027/0138

(57) ABSTRACT

An illustrated view of a welding mask view presenter device for presenting a safe view of a welding area is presented. The welding mask view presenter device is useful for providing a remote view of the welding area. Also, the welding mask view presenter device is useful preventing harmful sparks from hurting a welder's eyes and from inhaling smoke given off by the welding process. The welding mask view presenter device is useful for preventing a face of the welder from being exposed to the welding area and providing only hands of the welder from being exposed, though the hands are covered with welding gloves.

### FIELD OF THE INVENTION

This invention relates to welding. More particularly, it relates to a device to present a safe view of a welding area.

### BACKGROUND

A welding helmet is a type of headgear used when performing certain types of welding to protect the eyes, face and neck from flash burn, ultraviolet light, sparks, infrared light, and heat.

Welding helmets are most commonly used with arc welding processes such as shielded metal arc welding, gas tungsten arc welding, and gas metal arc welding. They are necessary to prevent arc eye, a painful condition where the cornea is inflamed. Welding helmets can also prevent retina burns, which can lead to a loss of vision. Both conditions are caused by unprotected exposure to the highly concentrated ultraviolet and infrared rays emitted by the welding arc. Ultraviolet emissions from the welding arc can also damage uncovered skin, causing a sunburn-like condition in a relatively short period of welding. In addition to the radiation, gasses or splashes can also be a hazard to the skin and the eyes.

Most welding helmets include a window covered with a filter called a lens shade, through which the welder can see to work. In most helmets, the window may be made of tinted glass, tinted plastic, or a variable-density filter made from a pair of polarized lenses. Different lens shades are needed for different welding processes.

All welding helmets are susceptible to damages such as cracks that can compromise the protection from ultraviolet and infrared rays. In addition to protecting the eyes, the helmet protects the face from hot metal sparks generated by the arc and from UV damage. When overhead welding, a leather skull cap and shoulder cover are used to prevent head and shoulder burns.

One of the major problems with the welding helmets is that the spark from the welding can cause damage to the retina of the welder. However, the welder does not always see the spark or all areas around the welding area.

Accordingly, and in light of the foregoing, it would be desirable to devise an camera that can see all areas around a welding area and provide a video or picture for a welder to see without being subject to damage to their retina or other burns that the welder may be subject normally. Furthermore, the device should allow for a distance from the welding and prevent smoke from being inhaled.

### BRIEF DESCRIPTION OF THE DRAWINGS

FIG. 1 is an illustrated view of an exemplary welding mask view presenter device.

FIG. 2A-FIG. 2C are illustrated views of a welding mask with the welding mask view presenter device showing in FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

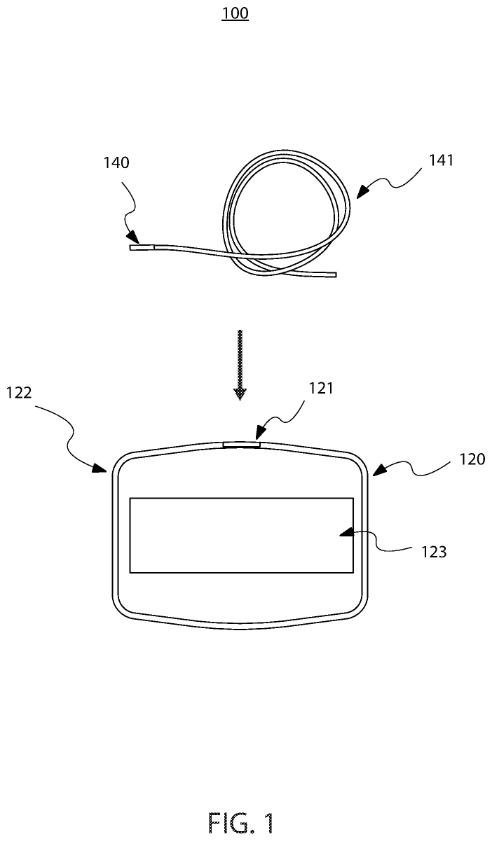

Referring to FIG. 1, an illustrated view of an exemplary welding mask view presenter device 100 for presenting a safe view of a welding area is presented. The welding mask view presenter device 100 is useful for providing a remote view of the welding area. Also, the welding mask view presenter device 100 is useful preventing harmful sparks from hurting a welder's eyes and from inhaling smoke given off by the welding process. The welding mask view presenter device 100 is useful for preventing a face of the welder from being exposed to the welding area and providing only hands of the welder from being exposed, though the hands are covered with welding gloves.

The welding mask view presenter device 100 has a display panel 120 and a camera 140. The camera 140 is coupled to a camera cable 141. The camera cable 141 is then coupled to a port 121 of the display panel 120 of the welding mask view presenter device 100.

The display panel 120 has an outside 122 and a display screen 123. The display screen 123 is preferably a light emitting diode (LED) screen, however other types of screens are hereby contemplated, including, but not limited to, liquid crystal display (LCD) screen, etc.

The camera 140 is movable, the camera 140 is movable by allowing the moving and manipulation of the camera cable 141 to be extended and to be placed in different locations.

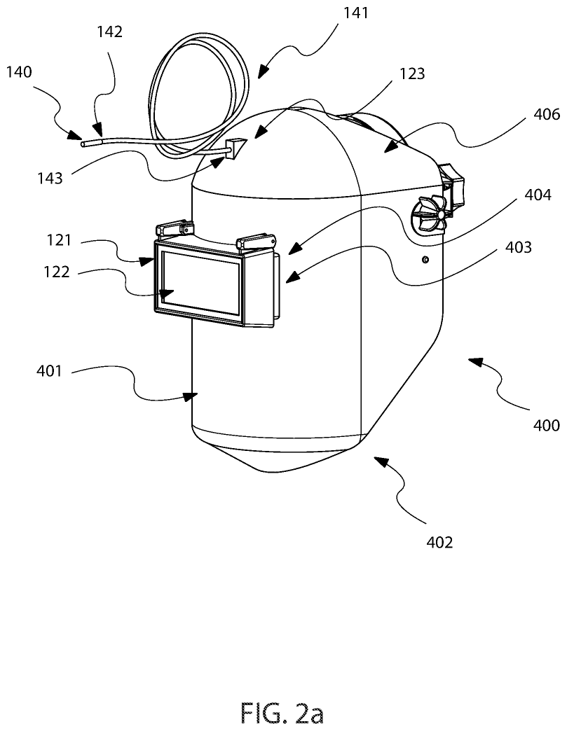

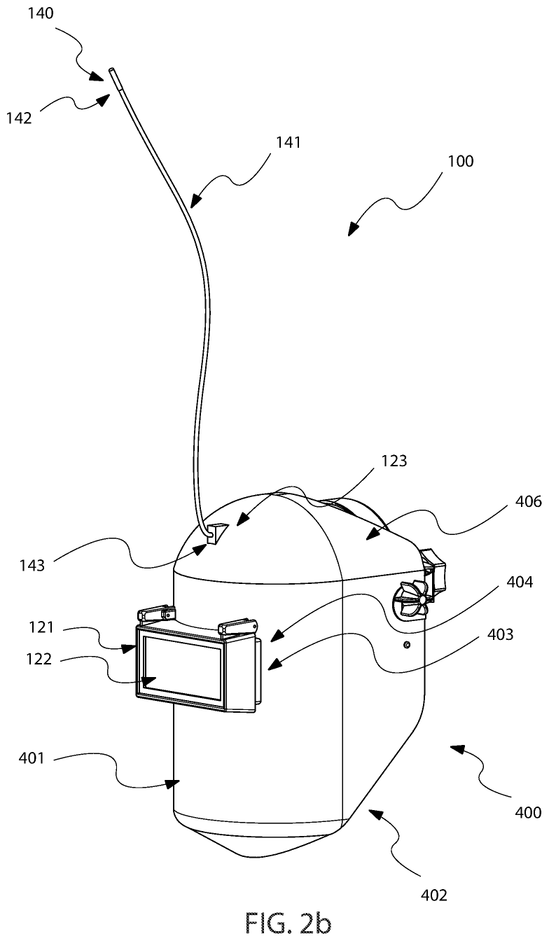

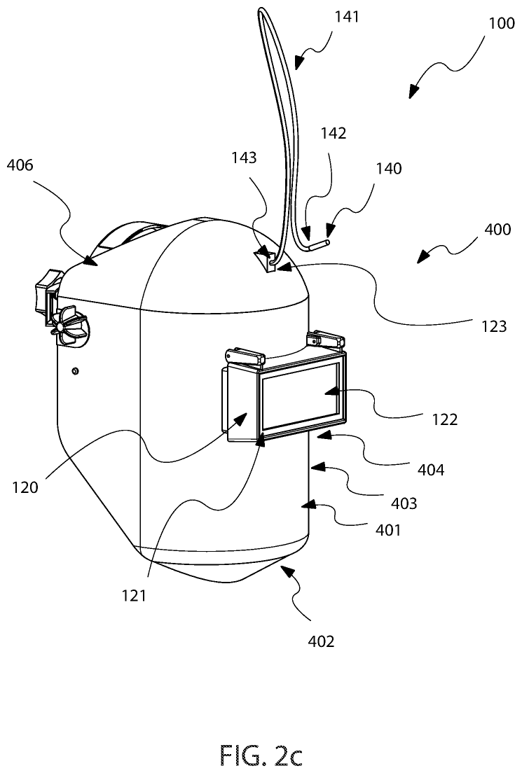

Moving now to FIG. 2A, FIG. 2B and FIG. 2C, the welding mask view presenter device 100 is coupled to a welding helmet 400. The welding helmet 400 has an outside 401, an inside 402, a front 403, a middle area 404.

The camera 140 is coupled to a first end 142 of the camera cable 141. The second end 143 is coupled to the port 123 through a top 406 of the welding helmet 400. The port 123 is preferably an universal serial bus (USB), however other types of ports are hereby contemplated, including, but not limited to, a pin port, etc. The port 123 is coupled to the display panel 120 of the welding mask view presenter device 100.

The display panel 120 has an outside 121 and a display screen 122. The display screen 122 is coupled to an opening 407 of the middle area 404 of the front 403 of the welding helmet 400. The display panel 120 is communicably coupled to the camera 140 by the port 123. The display screen 122 is viewable on the inside 402 of the welding helmet 400 and the outside 121 is viewable on the outside 401 of the welding helmet 400.

FIG. 2A shows the display panel 120 coupled to the middle area 404 of the front 402 of the welding helmet 400. The camera 140 is coupled to the port 123 through the welding helmet 400 by the camera cable 141.

In FIG. 2B, the camera cable 141 extended to view a particular welding area (not shown) while being coupled to the port 123 through the welding helmet 400. Images captured by the camera 140 are rendered on the display screen 122 of the display panel 120.

In FIG. 2C, the camera cable 141 has been further manipulated to capture images from the top 406 of the welding helmet 400 and the rendered or captured images from the camera 140 are displayed on the display screen 122 of the display panel 120 on the inside 402 of the welding helmet 400 for safe viewing of the welding area.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A welding mask view presenter device for providing a captured rendering of a welding area to a welder, the device consisting of:
a camera;
a camera cable, the camera cable has a first end and a second end, wherein the camera is coupled to the first end of the camera cable, and wherein the second end of the camera cable being coupled to a port;
a display panel, the display panel has an outside and an inside, wherein the camera captures an image and wherein the captured image is displayed on the inside of the display panel, and wherein the display panel being communicably coupled to the camera by the camera cable; and
wherein the display panel being coupled to a middle area of a welding helmet, wherein the display screen is visible on an inside of the welding helmet.

2. The device of claim 1, wherein the display panel is a light emitting diode (LED) panel.

3. The device of claim 1, wherein the port is a universal serial bus (USB) port.
